(19) United States
(12) Patent Application Publication
(10) Pub. No.: US 2025/0295870 A1
(43) Pub. Date: Sep. 25, 2025

(54) COMPOSITIONS AND METHODS FOR INHALABLE THERAPEUTICS

(71) Applicant: Inhalon Biopharma, Inc., Morrisville, NC (US)

(72) Inventors: Samuel K. LAI, Durham, NC (US); Thomas R. MOENCH, Baltimore, MD (US); Jeff T. HUTCHINS, Claremore, OK (US)

(21) Appl. No.: 18/869,268

(22) PCT Filed: May 23, 2023
(86) PCT No.: PCT/US2023/067373

§ 371 Date: Nov. 25, 2024

Related U.S. Application Data
(63) Continuation of application No. 17/889,141, filed on Aug. 16, 2022.
(60) Provisional application No. 63/233,661, filed on Aug. 16, 2021.
(60) Provisional application No. 63/345,019, filed on May 23, 2022.

(51) Int. Cl. A61M 15/00; A61K 9/00; C07K 16/00; C07K 16/10

(52) U.S. Cl. CPC A61M 15/0021; A61K 9/0078; A61M 15/009; C07K 16/00; C07K 16/1003; C07K 2317/24; C07K 2317/52

(57) ABSTRACT

Described herein are therapeutic inhaled antibodies and methods of delivering these therapeutic antibodies that may sustain a concentration of therapeutic inhaled antibody within the upper respiratory tract (URT) and the lower respiratory tract (LRT), as well as the blood, following even a single dose. The compositions and methods described herein may provide therapeutically-relevant levels of an inhaled antibody that is delivered by inhalation at a single dose delivered once per day or less frequently. These methods may result in a concentration in both the URT and LRT that is greater than a minimum threshold concentration having clinical relevance.

### CLAIM OF PRIORITY

This application claims priority to U.S. Provisional Patent Application No. 63/345,019, titled “COMPOSITIONS AND METHODS FOR INHALABLE THERAPEUTICS”, filed on May 23, 2022, and to U.S. patent application Ser. No. 17/889,141, filed on Aug. 16, 2022, which claims benefit of U.S. Provisional Patent Application No. 63/233,661, titled “METHODS AND APPARATUSES FOR DELIVERY OF AN AGENT TO THE LUNGS AND NASAL PASSAGES”, filed on Aug. 16, 2021, all of which are herein incorporated by reference in their entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

It is considered accepted that antibodies delivered by inhalation into the lungs, including Fc-conjugated antibodies, are rapidly cleared from the lungs. For example, Fc-conjugated proteins given by inhalation typically have Tmax in serum (i.e. time to reach Cmax) in the 10-20 hrs range, and thus have a much faster clearance (on the order of hours or minutes) in the lungs. Bitonti and Durmont, “Pulmonary administration of therapeutic proteins using an immunoglobulin transport pathway,” Advanced Drug Delivery Reviews, Volume 58, Issues 9-10, 31 Oct. 2006, Pages 1106-1118. Indeed, the therapeutic efficacy of inhaled drugs has long been believed to be limited by their rapid clearance in the lungs. Small solutes delivered to the lungs quickly diffuse across lung epithelia and penetrate the bloodstream within minutes. Peptides are rapidly transported to the systemic circulation as well but are significantly metabolized locally. As summarized by Loira-Pastoriza et al. (“Delivery strategies for sustained drug release in the lungs,” Advanced Drug Delivery Reviews, Volume 75, 30 Aug. 2014, Pages 81-91.): “Although macromolecules can be absorbed into the systemic circulation over several hours, they can be rapidly taken up by alveolar macrophages, they can be removed by the mucociliary escalator, and they can be metabolized locally as well. For instance, recombinant human deoxyribonuclease I is a 37 kDa glycoprotein which cleaves the DNA in respiratory secretions of cystic fibrosis patients and thus, lowers their viscosity. This glycoprotein is the mucolytic agent most widely used in the symptomatic treatment of cystic fibrosis. However, it is rapidly cleared from the human lungs: when the daily dose of 2.5 mg is inhaled, a concentration of 3 μg/ml is measured in sputum immediately after inhalation and it is reduced to 0.6 μg/ml after 2 h. Therefore, its once to twice daily administration provides limited therapeutic coverage to the patients.” Unfortunately, because a short residence time of drugs within the lungs also requires more frequent dosing and this is believed to jeopardize patient compliance. It is for instance recommended to inhale corticosteroids at least twice daily and short-acting β2-agonists up to 4-times daily.

Thus, it would be beneficial to provide compositions, and particularly mAb compositions, that may remain within the lungs for an extended period of time at clinically significant levels without being cleared. Such compositions and methods may provide numerous clinical and compliance benefits.

### SUMMARY OF THE DISCLOSURE

The present invention relates to therapeutic inhaled antibodies and methods of delivering these therapeutic antibodies to sustain a concentration of within the upper respiratory tract (URT) and the lower respiratory tract (LRT), as well as the blood, following even a single dose. Surprisingly, the compositions and methods described herein may provide therapeutically-relevant levels of an inhaled IgG antibody that is delivered by inhalation at a single dose delivered once per day or less frequently (e.g., between once per day and once per five days). These methods may result in a concentration in both the URT and LRT that is greater than a minimum threshold concentration having clinical relevance.

The persistence of the therapeutic mAb in the URT and LRT appears to be a result of the interaction of the core Fc region of the IgG backbone common to the therapeutic antibodies described herein (including, e.g., regdanvimab), regardless of the target-specific (variable region) of the individual mAbs. This may be because it is the Fc region that is interacting with the mucus and other components driving clearance of the mAb from the lungs. The effects described herein are particularly relevant to composition of mAb in which the IgG Fc regions are glycosylated in a manner that modulates the mucin interactions. For example, these compositions may include an Fc region that is glycosylated with a G0 glycosylation, e.g., comprising a biantennary core glycan structure of Manα1-6(Manα1-5)Manβ1-4GlcNAcβ1-4GlcNAcβ1 with terminal N-acetylglucosamine on each branch that enhances the trapping potency of the recombinant antibody in mucus.

For example, described herein are methods of treating a subject having, or at risk of having, a respiratory disorder, comprising administering by inhalation to the subject a formulation comprising a therapeutic antibody that binds to a respiratory virus in a dosing regimen comprising a dosing cycle of once per day or twice per day.

Thus, described herein are methods of treating a subject having, or at risk of having, a respiratory disorder, the method comprising administering, by inhalation, to the subject a formulation comprising a therapeutic human IgG monoclonal antibody (mAb) comprising a population of antibodies in which at least 40% are glycosylated with a G0 glycosylation pattern comprising a biantennary core glycan structure of Manα1-6(Manα1-3)Manβ1-4GlcNAcβ1-4GlcNAcβ1, wherein administering comprises administering in a dose of 0.02 μmol or more of the therapeutic human mAb no more than twice per day to achieve a concentration of greater than 20 ng/mL for the therapeutic human mAb in an upper respiratory tract (URT) and a concentration of greater than 100 ng/mL in a lower respiratory tract (LRT) for 12 hours or more after the dose.

In some examples a method of treating a subject having, or at risk of having, a respiratory disorder may include: maintaining a concentration of greater than 20 ng/ml of a therapeutic human IgG monoclonal antibody (mAb) that binds to a respiratory virus in an upper respiratory tract (URT) of the subject and a concentration of greater than 100 ng/ml in a lower respiratory tract (LRT) of the subject for more than 12 hours after a dose by administering, by inhalation, to the subject the dose of a therapeutic human IgG monoclonal antibody (mAb) comprising a population of antibodies in which at least 40% are glycosylated with a G0 glycosylation pattern comprising a biantennary core glycan structure of Manα1-6(Manα1-3)Manβ1-4GlcNAcβ1-4GlcNAcβ1, wherein administering the dose comprises administering 0.02 μmol or greater of the therapeutic human mAb no more than twice per day.

In any of these examples administering may comprise administering the dose no more than once per day.

In some examples, the therapeutic antibody may comprise at least 45% of the G0 glycosylation pattern (e.g., at least 50%, at least 55%, at least 60%, at least 65%, at least 70%, at least 80%, at least 85%, at least 90%, at least 95%, etc.).

In some examples the therapeutic antibody comprises an Fc sequence that is at least X % (e.g., 80%, 85%, 90%, 95%) homologous to the sequence of SEQ ID NO. 1 (e.g., human IgG1). For example, the therapeutic antibody comprises an Fc sequence that is at least 85% homologous to the sequence of SEQ ID NO. 1, including conservative peptide substitutions.

The therapeutic antibody may be regdanvimab. The dosing regimen may comprise a dosing cycle of twice per day over a period of two days to seven days. The dosing regimen may comprise a dosing cycle of every second day, every third day or every fourth day. The dosage regimen may comprise administering the dose of at least 10 mg of the therapeutic mAb. The dosage regimen may comprise administering the dose of between about 10 mg and 100 mg of the therapeutic mAb. In some examples administering comprises sustaining a release of the therapeutic mAb into the blood from the LRT over multiple days. Administering may comprise sustaining release of the mAb into the lungs and blood over at least two days.

The formulation may also comprise a pharmaceutically acceptable diluent, excipient, and/or carrier. In some examples the formulation further comprises one or more of: citrate, arginine, mannitol, sorbitol, trehalose.

The therapeutic antibody formulation may be administered to the subject via a nebulizer, such as a vibrating mesh nebulizer. In some examples the therapeutic antibody formulation is administered via inhalation or via direct instillation into an upper airway. The therapeutic antibody formulation may be self-administered by the subject.

The respiratory disorder may comprise a lower airway disorder. The respiratory disorder may comprise an upper airway disorder. In some examples the respiratory disorder comprises an inflammatory disorder. The respiratory virus may comprise a coronavirus. The respiratory virus may comprise severe acute respiratory syndrome coronavirus 2 (SARS-CoV-2). The respiratory virus may comprise respiratory syncytial virus (RSV). The respiratory virus may comprise one or more of: influenza, metapneumovirus, parainfluenza, (specific coronavirus). In some examples the respiratory virus comprises a paramyxovirus.

The formulation may comprise a second or more therapeutic agent in addition to the therapeutic antibody. The formulation may comprise the therapeutic mAb and a second therapeutic antibody, and the first therapeutic antibody and the second therapeutic antibody bind to the same virus, but do not compete for binding to the virus. In some examples the formulation comprises a second therapeutic antibody in addition to the first therapeutic antibody, further wherein the first antibody and the second antibody bind to different viruses. The formulation comprises a biologic in addition to the therapeutic mAb.

For example, a method of treating a subject having, or at risk of having, a respiratory disorder may include: maintaining a concentration of greater than 25 ng/ml of a therapeutic human IgG monoclonal antibody (mAb) that binds to a respiratory virus in an upper respiratory tract (URT) of the subject and a concentration of greater than 25 ng/ml in a lower respiratory tract (LRT) of the subject for more than 12 hours after the dose by administering, by inhalation, to the subject the dose of a formulation comprising a therapeutic human IgG monoclonal antibody (mAb) that binds to a respiratory virus, wherein administering comprises administering 0.02 μmol or greater of the therapeutic human mAb no more than twice per day.

Also described herein are methods of treating a subject having, or at risk of having, a respiratory disorder, the method comprising administering, by inhalation, to the subject a formulation comprising a therapeutic human IgG monoclonal antibody (mAb) that binds to a respiratory virus, wherein administering comprises administering a dose of 0.02 μmol or greater of the therapeutic human mAb no more than once per day to achieve a concentration of greater than 25 ng/ml for the therapeutic human mAb in an upper respiratory tract (URT) and a concentration of greater than 25 ng/ml in a lower respiratory tract (LRT) for more than 24 hours after the dose.

Also described herein are methods of treating a subject having, or at risk of having, a respiratory disorder, the method comprising administering, by inhalation, to the subject a formulation comprising a therapeutic human IgG monoclonal antibody (mAb) that is glycosylated with a G0 glycosylation pattern comprising a biantennary core glycan structure of Manα1-6(Manα1-3)Manβ1-4GlcNAcβ1-4GlcNAcβ1, wherein administering comprises administering in a dose of 0.02 μmol or more of the therapeutic human mAb no more than once per day to achieve a concentration of greater than 20 ng/mL for the therapeutic human mAb in an upper respiratory tract (URT) and a concentration of greater than 100 ng/mL in a lower respiratory tract (LRT) for more than 24 hours after the dose.

For example, a method of treating a subject having, or at risk of having, a respiratory disorder, may include maintaining a concentration of greater than 25 ng/ml of a therapeutic human IgG monoclonal antibody (mAb) that binds to a respiratory virus in an upper respiratory tract (URT) of the subject and a concentration of greater than 25 ng/ml in a lower respiratory tract (LRT) of the subject for more than 24 hours after a dose by administering, by inhalation, to the subject the dose of a formulation comprising a therapeutic human IgG monoclonal antibody (mAb) that binds to a respiratory virus, wherein administering comprises administering 0.02 μmol or greater of the therapeutic human mAb no more than once per day.

In some examples a method of treating a subject having, or at risk of having, a respiratory disorder, may include maintaining a concentration of greater than 20 ng/ml of a therapeutic human IgG monoclonal antibody (mAb) that binds to a respiratory virus in an upper respiratory tract (URT) of the subject and a concentration of greater than 100 ng/ml in a lower respiratory tract (LRT) of the subject for more than 24 hours after a dose by administering, by inhalation, to the subject the dose of a therapeutic human IgG monoclonal antibody (mAb) that is glycosylated with a G0 glycosylation pattern comprising a biantennary core glycan structure of Manα1-6(Manα1-3)Manβ1-4GlcNAcβ1-4GlcNAcβ1, wherein administering the dose comprises administering 0.02 μmol or greater of the therapeutic human mAb no more than once per day.

In any of the methods described herein the therapeutic antibody may be a therapeutic human IgG monoclonal antibody (mAb). In any of the methods described herein the therapeutic human IgG monoclonal antibody (mAb) is a human IgG1 mAb. In any of the methods described herein the therapeutic antibody comprises an Fc sequence that is at least X % (e.g., 80%, 85%, 90%, 95%) homologous to the sequence of SEQ ID NO. 1 (e.g., human IgG G1). For example, the therapeutic antibody may comprise regdanvimab. Alternatively, in any of these methods and compositions, the Fc sequence may be at least X % homologous to the sequence of one or more of SEQ ID NO.: 1, SEQ ID NO.: 2, SEQ ID NO.: 3, and/or SEQ ID NO.: 4.

In general, the subject may be any subject in need of the therapy. In particular, the subject may be an adult subject and young-adult subjects. As used herein, a young-adult subject may refer to any individual 12 and older.

In any of the methods described herein the therapeutic antibody may comprise an oligosaccharide that enhances the trapping potency of the recombinant antibody in mucus. For example, the therapeutic antibody may comprise a population of mAbs in which at least 40% comprises an oligosaccharide having a G0 glycosylation pattern comprising a biantennary core glycan structure of Manα1-6(Manα1-5)Manβ1-4GlcNAcβ1-4GlcNAcβ1 with terminal N-acetylglucosamine on each branch that enhances the trapping potency of the recombinant antibody in mucus.

In any of the methods described herein the dosing regimen may comprise a dosing cycle of once per day over a period of two days to seven days. The dosing regimen may comprise a dosing cycle of every second day, every third day or every fourth day. The dosing regimen may comprise administering a total of two, three, or four doses. The dosing regimen may comprise administering only a single dose. The dosage regimen may comprise administering the dose of at least 30 mg of the therapeutic mAb. The dosage regimen may comprise administering the dose of between about 30 mg and 90 mg of the therapeutic mAb.

In any of the methods described herein administering may comprise sustaining a release of the therapeutic mAb into the blood from the LRT over multiple days. Administering may comprise sustaining release of the mAb into the lungs and blood over at least two days.

In any of the methods described herein the formulation further comprises a pharmaceutically acceptable diluent, excipient, and/or carrier. For example, the formulation may further comprise one or more of: citrate, arginine, mannitol, sorbitol, trehalose. The therapeutic antibody formulation may be administered to the subject via a nebulizer. The therapeutic antibody formulation may be administered to the subject via a vibrating mesh nebulizer. The therapeutic antibody formulation may be administered to the subject via a nebulizer. The therapeutic antibody formulation may be administered via inhalation or via direct instillation into an upper airway. The therapeutic antibody formulation may be self-administered by the subject.

The respiratory disorder comprises a lower airway disorder. The respiratory disorder may comprise an upper airway disorder. The respiratory disorder may comprise an inflammatory disorder. The respiratory virus may comprise a coronavirus. The respiratory virus may comprise severe acute respiratory syndrome coronavirus 2 (SARS-CoV-2). The respiratory virus may comprise respiratory syncytial virus (RSV). The respiratory virus may comprise one or more of influenza, metapneumovirus, parainfluenza, (specific coronavirus). The respiratory virus may comprise a paramyxovirus.

In any of the methods described herein the formulation may comprise a second or more therapeutic agent in addition to the therapeutic antibody. The formulation may comprise the therapeutic mAb and a second therapeutic antibody, and the first therapeutic antibody and the second therapeutic antibody bind to the same virus, but do not compete for binding to the virus. The formulation may comprise a second therapeutic antibody in addition to the first therapeutic antibody, further wherein the first antibody and the second antibody bind to different viruses. The formulation may comprise a biologic in addition to the therapeutic mAb.

Also described herein are compositions (e.g., therapeutic human IgG monoclonal antibodies, and in particular, therapeutic human IgG monoclonal antibody (mAb) comprising a population of antibodies in which at least 40% are glycosylated with a G0 glycosylation pattern comprising a biantennary core glycan structure of Manα1-6(Manα1-3)Manβ1-4GlcNAcβ1-4GlcNAcβ1, for use in a method of treating any of the respiratory disorders described herein by performing any of the methods described. For example, described herein are therapeutic human IgG monoclonal antibody (mAb) comprising a population of antibodies in which at least 40% are glycosylated with a G0 glycosylation pattern comprising a biantennary core glycan structure of Manα1-6(Manα1-3)Manβ1-4GlcNAcβ1-4GlcNAcβ1, for use in a method of treatment of a respiratory disorder by administering, by inhalation, the therapeutic human IgG monoclonal antibody (mAb), wherein administering comprises administering in a dose of 0.02 μmol or more of the therapeutic human mAb no more than twice per day to achieve a concentration of greater than 20 ng/mL for the therapeutic human mAb in an upper respiratory tract (URT) and a concentration of greater than 100 ng/mL in a lower respiratory tract (LRT) for 12 hours or more after the dose.

Also described herein are therapeutic human IgG monoclonal antibody (mAb) comprising a population of antibodies in which at least 40% are glycosylated with a G0 glycosylation pattern comprising a biantennary core glycan structure of Manα1-6(Manα1-3)Manβ1-4GlcNAcβ1-4GlcNAcβ1, for use in a method of treatment of a respiratory disorder by maintaining a concentration of greater than 20 ng/ml of the therapeutic human IgG mAb in an upper respiratory tract (URT) of the subject and a concentration of greater than 100 ng/ml in a lower respiratory tract (LRT) of the subject for more than 12 hours after a dose by administering, by inhalation, the dose of the therapeutic human IgG mAb, wherein administering the dose comprises administering 0.02 μmol or greater of the therapeutic human mAb no more than twice per day.

These methods, and in particular the dosing regimen described herein, are both surprising and effective; prior to this work it was believed that much larger and/or more frequent dosing would be required, as the predicted clearance of the therapeutic (mAb) from the lungs was believed to be very fast (e.g., less than 30 minutes) for such compositions.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 is table 1, describing the demographics of patients enrolled in the study described in Example 1, showing the persistence of a therapeutic mAb having a human IgG Fc region that has been glycosylated (e.g., so that greater than 40% of the mAb is glycosylated) in the upper respiratory and lower respiratory tract (as seen in the serum level).

FIG. 2 is table 2 summarizing adverse events from the study described in Example 1. Side effects marked by a (*) occurred within 2-hours of completing nebulization; (cough, FEV1 decreased). Complications marked by (**) included contraceptive ID use.

FIG. 3 (left) shows an example of a process flow for the example method of treatment described in Example 1. FIG. 3 (right) shows an example of the study schema and sample collection timepoints used in Example 1.

FIGS. 4A-4C illustrate nasal fluid concentrations. FIG. 4A shows concentrations in single dose cohorts. FIG. 4B shows concentrations in daily multiple dose cohort (e.g., seven days of 90 mg). Arrows on the X axis indicated the 7 times of 90 mg dose administration in FIG. 4B.

FIG. 4C show a comparison of nasal concentrations between single dose and multiple dose cohorts. Average LLOQ for all nasal fluid samples is shown at 450 ng/g, but LLOQ varied by sample, depending on the mass of nasal fluid collected on swab, resulting in some detectable samples below the overall average LLOQ. The fractions below each timepoint represent the number of samples that fell below the LLOQ at that time.

FIGS. 5A-5B show serum IN-006 concentrations in single dose cohorts (FIG. 5A), and a multiple dose cohort (FIG. 5B, last dose administered at 144 h). Symbols plotted below the dashed LLOQ line at 25 ng/mL represent the number of samples in each group that were BLQ at each timepoint.

FIG. 6 is a schematic illustrating one example of a method as described herein.

### DESCRIPTION

Described herein are methods, compositions and apparatuses (e.g., devices, systems, etc.) useful for treating a subject having, or at risk of having, a respiratory disorder. In some embodiments, provided are methods of administering a therapeutic antibody to treat a subject having or at risk of having a respiratory disorder affecting the upper respiratory tract (upper airway) or the lower respiratory tract (lower airway). Methods provided herein may be especially useful for treating a subject having or at risk of having a respiratory disorder affecting both the upper respiratory tract (upper airway) and the lower respiratory tract (lower airway). Applicant has surprisingly and unexpectedly found using the methods, compositions, and apparatuses described herein the ability to achieve prolonged coverage with a therapeutic antibody that allows for an infrequent or episodic dosage regimen frequency (such as one-time delivery, once-daily delivery not more than twice-daily delivery).

The term “antibody” (Ab) refers to an immunoglobulin molecule that specifically binds to, or is immunologically reactive with, a particular antigen. Basic antibodies have a Y-shape with a stem region and two arm regions and can be classified into different categories, called isotypes, based on features found in the antibody stem region. Basic antibodies are heterotetrameric glycoproteins composed of two identical light (L) chains and two identical heavy (H) chains. Each of the four chains has a variable (V) region at its amino terminus, which contributes to the antigen-binding site, and a constant (C) region, which determines the isotype. Experimentally, antibodies can be cleaved with the proteolytic enzyme papain, which causes each of the heavy chains to break, producing three separate subunits. Two of the units are composed of a light chain and a fragment of the broken heavy chain approximately equal in mass to the light chain. Each of these two units can separately bind antigen and are called Fab fragments (i.e., the “antigen binding” fragments). By some estimates, humans may be capable of producing as many as 10¹⁸, or one quintillion, distinct antibodies and each antibody would have unique Fab fragments. The third of the three units is composed of two equal segments of the heavy chain. This third unit is typically not involved in antigen binding but is important in later processes in the body involved in ridding the body of the antigen. In contrast to the Fab fragments, the third unit from the antibody typically has one of only five types of physicochemical properties and thus is called the Fc fragment (i.e., the “crystalalizable” fragment). The types of human antibodies containing one of the five types of Fc fragments are referred to as IgA, IgD, IgE, IgG, and IgM isotypes. These isotypes also may have several subclasses. For example, IgG antibodies in humans may be further divided into the subclasses IgG1, IgG2, IgG3, and IgG4. IgG antibodies in mice can be further subdivided into the subclasses IgG1, IgG2a, IgG2b and IgG3. Types and modified forms of antibodies can be produced by methods known in the art and include polyclonal, monoclonal, genetically engineered, bifunctional, chimeric antibodies, humanized antibodies, heteroconjugate antibodies (e.g., bispecific antibodies, diabodies, triabodies, and tetrabodies), and antigen binding fragments of antibodies or single chain antibodies, including e.g., Fab′, F(ab′)₂, Fab, Fv, rIgG, and scFv fragments (e.g., a single chain Fv) fragment including a VL domain linked to a VH domain by a linker.

A “blocking” antibody (also referred to as an “antagonist” antibody) is an antibody that inhibits or reduces the biological activity of the antigen it binds. In some embodiments, blocking antibodies or antagonist antibodies substantially or completely inhibit the biological activity of the antigen.

“Carriers” are generally designed to interact with, and enhance the properties, of active pharmaceutical ingredients (APIs) (e.g., antibodies). Carriers are generally safe and nontoxic to the subject and cells being exposed thereto at the dosages and concentrations employed. An example of a physiologically acceptable carrier is an aqueous pH buffered solution, such as a saline solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; salt-forming counterions such as sodium; and/or nonionic surfactants such as TWEEN™ polyethylene glycol (PEG), and PLURONICS™.

The term “Cmax” refers to a standard pharmacokinetic measure used to determine drug dosing. Cmax is the peak (highest) concentration maximum (or peak) concentration that a drug achieves in a specified compartment or test area of the body (e.g., blood, serum, nasal cavity, etc.) after the drug has been administered and before the administration of a subsequent (second) dose.

The term “effective amount” (or “therapeutically effective amount”) is at least the minimum agent concentration required to cause a measurable improvement or prevention of a particular disorder. An effective amount herein may vary according to factors such as the particular disorder (e.g., disease state), age, sex, and weight of the subject, and the ability of the agent (e.g., antibody) to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity of, or delaying the onset of the disorder (disease), including biochemical, histological and/or behavioral symptoms of the disorder (disease), its complications and intermediate pathological phenotypes presenting during development of the disorder (disease). For therapeutic use, beneficial or desired results include clinical results such as decreasing one or more symptoms resulting from the disorder (disease), increasing the quality of life of those suffering from the disorder (disease), decreasing the dose of other medications required to treat the disorder (disease), enhancing effect of another medication such as via targeting, delaying the progression of the disease, and/or prolonging survival. An effective amount can be administered in one or more administrations. For purposes herein, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

The term “excipient” refers to substances in a formulation other than the active pharmaceutical ingredient(s) (e.g., antibody). Examples of excipients include antioxidants, buffering agents, emulsifiers, penetration enhancers, preservatives, release controlling reagents, and viscosity modifiers.

The term “humanized antibodies” or “humanized” forms of non-human (e.g., murine) antibodies refers to chimeric antibodies that contain minimal sequence derived from non-human immunoglobulin. In some embodiments, a humanized antibody is a human immunoglobulin (recipient antibody) in which residues from a hypervariable region of the recipient are replaced by residues from a hypervariable region of a non-human species (donor antibody) such as mouse, rat, rabbit or non-human primate having the desired specificity, affinity, and/or capacity. The humanized antibody can also comprise at least a portion of an Fc, typically that of a human immunoglobulin consensus sequence. Methods of antibody humanization are known in the art. In some instances, framework (“FR”) residues of the human immunoglobulin are replaced by corresponding non-human residues. Furthermore, humanized antibodies may comprise residues that are not found in the recipient antibody or in the donor antibody.

The term “ka” (M⁻¹sec⁻¹) is intended to refer to the association rate constant of a particular antibody-antigen interaction. The term “K_{A}” (M), as used herein, is intended to refer to the association equilibrium constant of a particular antibody-antigen interaction.

The term “kd” (sec⁻¹), as used herein, is intended to refer to the dissociation rate constant of a particular antibody-antigen interaction. This value is also referred to as the off value. The term “K_{D}” (M⁻¹), as used herein, is intended to refer to the dissociation equilibrium constant of a particular antibody-antigen interaction.

In certain embodiments, antibodies of the disclosure are monoclonal antibodies. The term “monoclonal antibody” as used herein includes but is not limited to antibodies produced through hybridoma technology. The term “monoclonal antibody” refers to an antibody that is derived from a single clone, including any eukaryotic, prokaryotic, or phage clone, and not the method by which it is produced. Monoclonal antibodies useful in connection with the present disclosure can be prepared using a wide variety of techniques known in the art including the use of hybridoma, recombinant, and phage display technologies, or a combination thereof. The antibodies of the disclosure include chimeric, primatized, humanized, or human antibodies.

The term “nebulizer” refers to a device configured to change a medication (formulation) from a liquid to an aerosol or suspension of fine particles or droplets (also referred to herein as a mist) and to deliver the aerosol to a subject for breathing the aerosol into the lungs. Nebulizer devices include jet nebulizers, mesh nebulizers, and ultrasonic nebulizers. Nebulizers can also be heated or refillable. A jet nebulizer (also sometimes referred to as a compressor, nozzle, pneumatic, or venturi nebulizer) uses a compressed gas (such as air or oxygen) to form an aerosol. For example, a nebulizer reservoir can be filled with medication (formulation). Compressed gas can be applied to an inlet of the reservoir and traveling at high velocity, exit through a narrow orifice, creating an area of low pressure at the outlet. The resulting pressure differential causes fluid from the reservoir to be drawn up into and out of reservoir. The fluid can then be shattered into droplets of various sizes by the nebulizer walls or internal baffles. An ultrasonic nebulizer uses high-frequency vibrations such as 2-3 million/second from a piezo-electric vibrator. The vibrations can be transferred through a cooling water tank to the medication (formulation) to form an aerosol. A mesh nebulizer uses a very fine mesh to form a mist. A vibrating element pushes a medication (formulation) through microscopic holes in a membrane (e.g., a mesh). This generates an aerosol of small droplets. The phrase “pharmaceutically acceptable” indicates that the substance or composition must be compatible chemically and/or toxicologically, with the other ingredients comprising a formulation, and/or the mammal being treated therewith.

The term “peak level” refers to the highest concentration in an individual's body of a therapeutic agent (e.g., antibody).

The term “pharmaceutically acceptable salt” refers to pharmaceutically acceptable organic or inorganic salts of a compound of the invention. Exemplary salts include, but are not limited, to acetate, bisulfate, bromide, chloride, citrate, iodide, nitrate, oleate, oxalate, pantothenate, sulfate, phosphate, acid phosphate, isonicotinate, lactate, salicylate, acid citrate, tartrate, tannate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, pamoate (i.e., 1,1′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

The term “specific binding” of an antibody refers to antibody binding to a predetermined antigen. Typically, the antibody binds with an affinity corresponding to a K_{D} of about 10⁻⁸ M or less and binds to the predetermined antigen with an affinity (as expressed by K_{D}) that is at least 10 fold less, and preferably at least 100 fold less than its affinity for binding to a nonspecific antigen (e.g., BSA, casein) other than the predetermined antigen or a closely-related antigen. Alternatively, the antibody can bind with an affinity corresponding to a K_{A} of about 10⁶ M⁻¹, or about 10⁷M⁻¹, or about 10⁸M⁻¹, or 10⁹M⁻¹ or higher, and binds to the predetermined antigen with an affinity (as expressed by K_{A}) that is at least 10 fold higher, and preferably at least 100 fold higher than its affinity for binding to a non-specific antigen (e.g., BSA, casein) other than the predetermined antigen or a closely-related antigen.

The term “treatment” refers to clinical intervention designed to alter the natural course of the individual or cell being treated during the course of clinical pathology or to prevent a course of clinical pathology from occurring. Desirable effects of treatment include decreasing the rate of disease progression, ameliorating or palliating the disease state, and remission or improved prognosis. For example, an individual is successfully “treated” if one or more symptoms associated with a respiratory disorder are ameliorated, reduced, eliminated, or prevented, such as aches, bronchitis, chills, confusion, coughing, death, diarrhea, difficulty breathing, fatigue, fever, headache, inflammation, pale/gray/blue-colored skin/lips/nail beds, pneumonia, rhinorrhea (nasal congestion), shortness of breath, sneezing, sore throat, vomiting, weakness.

The term “trough level” refers to the lowest concentration in an individual's body of a therapeutic agent while the therapeutic agent is in a therapeutic range or of a concentration of therapeutic agent concentration prior to giving a further dose of the therapeutic agent.

The term “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites. Therapeutic antibody can be administered to the upper respiratory tract (also referred to as the upper airway) and/or the lower respiratory tract (also referred to as the lower airway). In some embodiments, an antibody is administered to both the upper respiratory tract and the lower respiratory tract. The upper airway includes the nose and nasal passages, paranasal sinuses, the oral cavity, the pharynx, and the portion of the larynx above the vocal cords, while the lower airway is further divided into the conducting zone and respiratory zone. The conducting zone is formed by the portion of the larynx below the vocal cords, trachea, and within the lungs, the bronchi and bronchioles. The respiratory zone is formed by the respiratory bronchioles, alveolar ducts, and alveoli. Therapeutic antibody can be administered to the upper and/or lower airway by dry powdered inhalers (DPI), injection, metered dose inhalers, nasal sprays, or nebulizers. A nebulizer is a drug delivery device that turns liquid medicine, such as the antibody composition described herein, into fine droplets (aerosol or mist) that gets inhaled into a subject's lungs, such as through a face mask or mouthpiece. Nebulizers include jet nebulizers, ultrasonic nebulizers, and mesh nebulizers. Examples of nebulizers that can be used to administer a therapeutic antibody include the Acorn and Acorn II® nebulizer (Vital Signs), AERx nebulizer (Aradigm), AeroDose nebulizer (AeroGen Inc., Mountain View, CA), Respimat nebulizer (Boehringer, Germany), and UltraVent™ nebulizer (Mallinckrodt). In some embodiments, a nebulizer used to practice the methods herein is a non-jet nebulizer and/or a non-ultrasonic nebulizer. In some embodiments, a nebulizer used to practice the methods herein is a mesh nebulizer. A mesh nebulizer can be gentler and less disruptive to antibody structure. Antibody potency is highly dependent upon their higher-order structure or conformation. Antibodies are proteins that undergo multiple stages of complicated protein folding during formation to generate their complex higher-order structure. These stages are primary, secondary, tertiary, and quaternary. The primary stage is the sequence of amino acids held together by peptide bonds. The secondary stage is the protein beginning to fold up (to form, e.g., alpha helices or beta-pleated sheets). Hydrogen bonds form between amino acids. The tertiary stage is the antibody tertiary structure when the protein folds into its 3D structure that relates to its function. The tertiary structure is held together by various non-covalent interactions between side groups, including ionic interactions, disulfide bridge formation, hydrophobic interactions, van der Waals forces, and hydrogen bonds. The quaternary stage is when single peptides bond to other peptides, such as when heavy and light chains join together. Antibodies can be sensitive to degradation due to many types of physical and chemical stress, such as freezing, heating, agitation, oxidation, and pH changes. Any of the compositions herein may include a pharmaceutically acceptable diluent, excipient, or carrier.

The nebulizers described herein may be configured to generate particle sizes with a predetermined range. The particle size range may be within a preferred range for deposition within both the lungs and the nasal passages using the methods described herein. Particles outside of the desired range may not be delivered within the nasal passages with the desired distribution pattern or level. For example, in any of the methods described herein, operating the nebulizer to continuously form particles containing the agent may comprise forming particles of average particle or droplet size (commonly defined as median mass aerodynamic diameter, MMAD) in the range from about 0.1 to about 200 microns (such as between about 1 to 10 microns, between about 2 to 7 microns, between about 2 to 20 microns, between about 10-40 microns, between about 20-60 microns, between about 30-70 microns, between about 40-80 microns, between about 50-90 microns, between about 60-100 microns, between about 70-110 microns, between about 80-120 microns, between about 90-130 microns, between about 100-150 microns, between about 125-200 microns, etc.). For example, operating the nebulizer to continuously form particles containing the agent comprises forming particles of average particle or droplet size in the range from about 2 to 7 microns. In some examples the method described herein may be used with two distributions of particle sizes, including smaller and larger particle sizes.

In some variations, inhaled respiratory medications can be administered using a device called a metered dose inhaler, or MDI. The MDI is a pressurized canister of medicine in a plastic holder with a mouthpiece. When sprayed, it can give a reliable, consistent dose of medication.

The present disclosure provides therapeutic regimens involving administering one or more therapeutic agents, including one or more than one antibody to a subject having or at risk of having a disorder (a respiratory disorder). A dosing regimen (e.g., a therapeutic regimen) for administering can vary depending on the upon the age and the size of a subject to be administered, target disease, antibody particulars, conditions/health/disease condition, route of administration. A dosing regimen can be more than once a day, but in general will be once a day or twice per day. In some variations, a dosing regimen can include more frequent therapeutic agent administration, such as three times per day, four times per day, etc. In some embodiments, a dosing regimen is administered once a day for only one day (i.e., only one dose). In some embodiments, a dosing regimen can continue for one day to indefinitely. In some embodiments, a dosing regimen is continued for two days, three days, four days, five days, six days, seven days, etc. or longer. In some embodiments, a dosing regimen can have regular administration intervals, such as every day, every second day, every third day, every week, every two weeks, every month, etc. or between these administration intervals. In some embodiments, a dosing regimen can be a non-variable dose regimen (e.g., each dose is the same amount) or a variable-dose regimen (different doses are different amounts, such as a larger amount of antibody in a first dose and less, such as half as much in a subsequent dose, one-third as much, one-quarter as much, etc.). A once per day delivery regimen may be convenient and facilitate successful adherence to the regiment. While a more frequent than a once per day delivery regimen may be less convenient, there may be an advantage to more frequent than once a day delivery. For example, for some therapeutic agents, such as expensive monoclonal antibodies, the total amount of therapeutic agent delivered in two doses can be less than the amount of therapeutic agent that would be delivered in a single once per day dose, and a two (or optionally more) dose per day delivery regimen can lead to lower cost. As illustrated in Example 4, to minimize trough levels from getting too low and thus losing efficacy, either a single high dose once a day, or a substantially lower dose twice a day can be administered. Each of these two doses can be so much lower that it more than makes up for inconvenience of administering doses twice per day. Thus, providing once daily vs twice daily (three times, etc.) dosing can balance convenience and efficient use of the antibody, such as, for example, depending on antibody production costs, accessibility to delivery options (e.g., self-administration, medical professional availability for therapeutic agent administration, use of a medical facility for therapeutic agent administration, etc.).

Specific antibodies and their EC50 A variety of drug agents may benefit from the nebulized delivery methods and apparatuses described herein. In particular, these methods and apparatuses may benefit drug agents for treating respiratory disorders affecting at least one of the upper and lower respiratory tracts though typically can benefit (treat) both the upper and lower respiratory tracts. In some embodiments, the methods and apparatuses may be useful for alleviating symptoms in the upper respiratory tract (e.g., from respiratory infections) as well as treating the lower respiratory tract, which is typically more relevant for hospitalization and other serious adverse outcomes. These methods and apparatuses may be particularly effective in delivery drug agents that are configured as mucosal binding and/or trapping agents. For example, the methods and apparatuses described herein may be particularly useful and/or effective when the drug agent is a recombinant antibody comprising an oligosaccharide having a G0 glycosylation pattern comprising a biantennary core glycan structure of Manα1-6(Manα1-3)Manβ1-4GlcNAcβ1-4GlcNAcβ1 with terminal N-acetylglucosamine on each branch that enhances the trapping potency of the recombinant antibody in mucus. In some examples the drug agent comprises a recombinant antibody comprising a human or humanized Fc region, wherein the recombinant antibody comprises a population of antibodies in which at least 20% (e.g., 20% or more, 25% or more, 30% or more, 35% or more, 40% or more, etc.) comprise an oligosaccharide having a G0 glycosylation pattern comprising a biantennary core glycan structure of Manα1-6(Manα1-3)Manβ1-4GlcNAcβ1-4GlcNAcβ1 with terminal N-acetylglucosamine on each branch that enhances the trapping potency of the recombinant antibody in mucus.

The methods and devices described herein can be used with for treating a subject having, or at risk of having, a respiratory disorder by administering one or more therapeutic antibodies. Examples of antibodies that can be used with the methods and devices herein include anti-cluster of differentiation 39 (CD39) antibody having an antibody or its antigen-binding fragment capable of specifically binding to human cluster of differentiation 39 (CD39) and as described in US20210388105A1. The antibody or its antigen-binding fragment is capable of specifically binding to human CD39 at half maximal effective concentration (EC50) of ≯10⁻⁸ M as measured by fluorescence-activated cell sorting (FACS) assay. CD39 has been implicated in pathogenesis of cigarette smoke-induced lung inflammation in patients and preclinical mouse models.

Another antibody that can be used with the methods and devices herein is anti-influenza B antibody as disclosed in US20210171612A1 (Regeneron Pharmaceuticals Inc., Tarrytown, NY). The anti-influenza B antibody can be an IgG1 or an IgG4 antibody that confers an increase in protection from influenza B virus in an animal (e.g., a mammal) when administered either subcutaneously or intravenously and/or when administered prior to infection, or after infection with influenza B virus and may reduce symptoms of headache, fever, aches, rhinorrhea (nasal congestion), chills, fatigue, weakness, sore throat, cough, shortness of breath, vomiting, diarrhea, pneumonia, bronchitis, and/or death. The anti-influenza B antibody binds to influenza B HA with an EC50 of less than about 10−9 M.

Another antibody that can be used with the methods and devices herein is anti-PCRV antibody as disclosed in US20200392210A1 (Regeneron Pharmaceuticals Inc. Tarrytown, NY). The anti-PCRV antibody can bind P. aeruginosa's V-tip protein (PcrV) and inhibit or neutralize the activity of the bacterial type 3 secretion system (T3SS) in P. aeruginosa. It is thought that the antibodies are useful for blocking translocation of toxins from the bacteria to the host cell and/or for preventing death of the host cells. The anti-PCRV antibodies may function by blocking pore-mediated membrane permeability in the host cell. The anti-PCRV antibody may bind to full length PcrV with an EC50 of less than about 10−⁸M. As disclosed in US20200392210A1, a patient at greater risk for P. aeruginosa infection can be a patient with cystic fibrosis, with diabetes, on a mechanical ventilator, undergoing surgery, with tuberculosis, with HIV, with a compromised immune system, with neutropenia, with an indwelling catheter, after physical trauma, with burns, in an intensive care unit, who is bedridden, with malignancy, with chronic obstructive pulmonary disease, in a long-term care health facility, or who is an intravenous drug user.

Another antibody that can be used with the methods and devices herein is anti-PD1 antibody (Apollomics Inc., Foster City, CA) as disclosed in U.S. Ser. No. 10/981,994B2. The anti-PD-1 antibody can be a humanized antibody wherein the anti-PD-1 antibody has a PD-1 binding EC50 of about 200 ng/ml or less or about 150 ng/mL or less or about 100 ng/mL or less or about 80 ng/ml or less or about 60 ng/mL or less, as measured by ELISA or FACS. The anti-PD-1 antibodies and fragments thereof provided bind to PD-1 on T cells, disrupting the PD-1/PD-L1 interaction and resulting in an increase in T cell activation. U.S. Ser. No. 10/981,994B2 discloses that IgG1 and IgG4 versions of the humanized 7A4 and 13F1 antibodies were produced. Anti-PD-1 antibody may be useful for treating infectious diseases, including respiratory disease, such as candidiasis, candidemia, aspergillosis, streptococcal pneumonia, streptococcal skin and oropharyngeal conditions, gram negative sepsis, tuberculosis, mononucleosis, influenza, respiratory illness caused by Respiratory Syncytial Virus, malaria, schistosomiasis, and trypanosomiasis. Another antibody that can be used with the methods and devices herein is bamlanivimab/etesevimab (created by Eli Lilly). Eli Lilly's monoclonal antibody bamlanivimab (also known as LY-CoV555, aka LY3819253) was originally derived from the blood of one of the first U.S. patients who recovered from COVID-19. It is a recombinant neutralizing monoclonal antibody directed against the SARS-CoV-2 spike protein. Eli Lilly's etesevimab (LY-CoV016, aka JS016, aka LY3832479) is a monoclonal antibody directed against the SARS-CoV-2 surface spike protein's receptor binding domain. Another antibody that can be used with the methods and devices herein is Bebtelovimab. The monoclonal antibody Bebtelovimab (Eli Lilly, Indianapolis, IN) has been used for treatment of mild to moderate COVID-19. Bebtelovimab binds to the SARS-CoV-2 spike protein. Bebtelovimab was administered as a single 175 mg intravenous injection over at least 30 seconds. Bebtelovimab is a human immunoglobulin G-1 (IgG1 variant) monoclonal antibody having 2 identical light chain polypeptides composed of 215 amino acids each and 2 identical heavy chain polypeptides composed of 449 amino acids. It has been produced by a Chinese Hamster Ovary (CHO) stable bulk culture or cell line with a molecular weight of 144 kDa. Bebtelovimab is a recombinant neutralizing human IgG1λ monoclonal antibody (mAb) to the spike protein of SARS-CoV-2 and is unmodified in the Fc region. Bebtelovimab was reported to bind the spike protein with a dissociation constant KD=0.046 to 0.075 nM and block spike protein attachment to the human ACE2 receptor with an IC50 value of 0.39 nM (0.056 mcg/mL). Another antibody that can be used with the methods and devices herein is casirivimab/imdevimab (created by Regeneron, brand name REGEN-COV). Regeneron's REGEN-COV (previously known as REGN-CoV2 or REGEN-CoV2) is two antibodies that bind to different regions of the SARS-CoV-2 spike protein receptor binding domain: casirivimab (REGN10933) and imdevimab (REGN10987). Another antibody that can be used with the methods and devices herein is regdanvimab or CT-P59 (Celltrion). Regdanvimab is a recombinant human monoclonal antibody targeted against the receptor binding domain (RBD) of the spike protein of severe acute respiratory syndrome coronavirus 2 (SARS-CoV-2) It is a recombinant monoclonal antibody expressed in CHO-K1 cells. The dose-dependent binding of a CT-P59 clinical batch to RBD was found as shown in the half-maximal effective concentration (EC50) of CT-P59 to SARS-CoV-2 RBD protein was 4.4 ng/ml.

Another antibody that can be used with the methods and devices herein is sotrovimab (created by Vir Biotechnology/GSK). Sotrovimab (formerly VIR-7831) has been reported to bind to a highly conserved epitope of the receptor binding domain of SARS-CoV-2 viral spike protein. Other antibodies that can be used with the methods and devices herein is Tixagevimab/cilgavimab (created by AstraZeneca, AZD7442, brand name Evusheld™) Evusheld™) emergency-authorized as pre-exposure prophylaxis against COVID-19 among immunocompromised individuals or those who cannot be vaccinated or mount post-vaccination immune response. AZD7442 contains two monoclonal antibodies, tixagevimab (AZD8895) and cilgavimab (AZD1061), which target the receptor binding domain of the SARS-CoV-2 spike protein.

The methods and devices described herein may be especially useful for treating respiratory disorders. In some embodiments, the present disclosure provides methods to treat (ameliorate, alleviate, or reduce), the severity, duration, or frequency of occurrence, of at least one symptom of a disorder or to prevent a disorder altogether. A symptom that the methods of the present disclosure may treat or prevent can be one or more of headache, fever, aches, rhinorrhea (nasal congestion), chills, fatigue, weakness, sore throat, cough, shortness of breath, vomiting, diarrhea, pneumonia, bronchitis, inflammation, and death. Inflammation and other symptoms can be acute or can be chronic. Causative agents of a disorder can include one or more of disease, environmental factors, genetic factors, illness, infection, pathogens, toxins, and or trauma. Pathogens can include archaebacteria, eubacteria, fungi, protists, and/or viruses. In some embodiments, a pathogen is a respiratory pathogen and can be a bacteria (such as Haemophilus (Haemophilus influenzae, Haemophilus influenzae (Type B)), Morazella (Morazella catarrhalis), Pseudomonas (Pseudomonas aeruginosa), Staphylocossus (Staphylocossus aureus), (Streptococcus (Streptococcus pneumoniae; Streptococcus pyogenes), fungi (such as Aspergillis, Blastomyces, Candida, Cryptocossus, Histoplasma, mold, yeast, Zygomycetes), or a virus (such adenovirus, coronavirus, influenza virus, metapneumovirus, Middle East respiratory syndrome coronavirus (MERS-CoV), parainfluenza virus, respiratory syncytial virus, severe acute respiratory syndrome coronavirus (SARS-CoV), rhinovirus, SARS-CoV-2). Other respiratory disorders that may be treated using the methods and devices herein include asthma, rhinitis, lung fibrosis, cystic fibrosis and chronic obstructive pulmonary disease.

For treatment of respiratory disorders, one (or more than one) additional therapeutic agents can suitably be used in combination with the antibodies described herein. Additional therapeutic agents can be a short-acting beta-agonist such as a cathecholamine or non-cathecholamine agent. Examples include, but are not limited to albuterol (ProAir HFA, Proventil HFA, Ventolin HFA), bitolterol, carbuterol, clenbuterol, epinephrine (Asthmanefrin, Primatene Mist), levalbuterol (Xopenex HFA), metaproterenol (Alupent), pirbuterol (Maxair), procaterol, terbutaline (Brethine), or other bronchodilators. Additional therapeutic agents can be anticholinergics, such as ipratropium (Atrovent) or other mucus-lessening agents. Additional therapeutic agents can be corticosteroids such as methylprednisolone and prednisone or other swelling-reducing agents. For treatment of respiratory disorders, anti-inflammatory agents can suitably be used in combination with the antibodies of the disclosure. Anti-inflammatory agents include, but are not limited to, acetaminophen, aspirin, dexamethasone, diphenhydramine, meperidine, mesalazine, asacol, codeine phosphate, benorylate, fenbufen, naprosyn, diclofenac, etodolac and indomethacin, and ibuprofen.

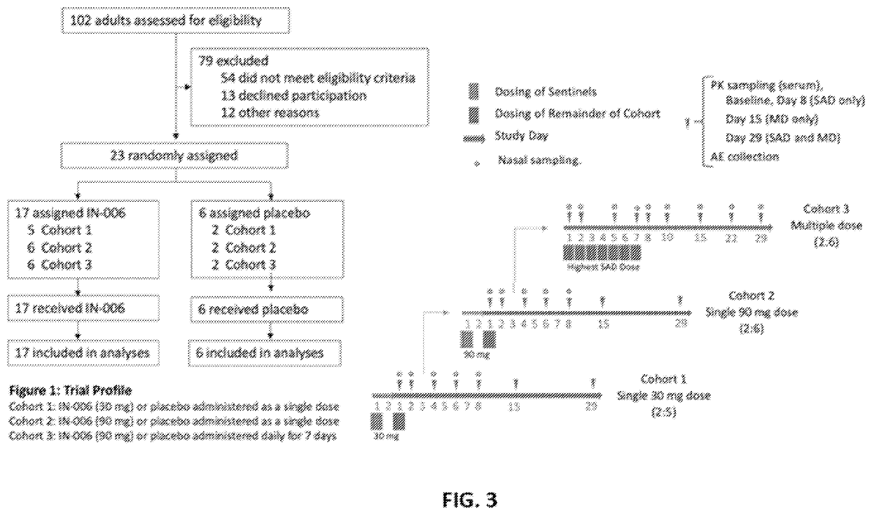

In one example, shown by the schematic in FIG. 3 the method 100 may include instructing and/or guiding the patient to administer a nebulization dose by first (optionally) sitting in an upright position 101. The patient may then activate the nebulizer to continuously provide a nebulized drug agent 103. For example, the method may include guiding the patient to press the on/off button on the nebulizer to start the treatment (e.g., in some examples the button will turn green, and mist will appear at the mouthpiece and/or the back of the nebulizer). The method may then guide the patient to hold the mouthpiece of the nebulizer between the lips 105, including holding the mouthpiece with the teeth and/or lips, and sealing the lips around the mouthpiece.

The method may then include activating (e.g., triggering) a first indicator to coach or guide the patient in inhaling the nebulized drug agent through the mouth 107. The first indicator may be, for example, a light (LED or LEDs), tone, message, countdown, etc. that remains on while the patient inhales to guide them to inhale deeply to draw the nebulized agent in through the mouth. The indicator may be a count (e.g., counting up or down). The indicator may be triggered automatically, including by a controller with or without input from the patient. In some examples the patient may manually trigger the start (activation) of the first indicator. Alternatively, in some examples the first indicator may be triggered upon sensing (e.g., in the nebulizer and/or in a dose guide apparatus) that the patient has started inhaling through their mouth. The first indicator may remain on for the inhalation duration of, e.g., 4 seconds or more (e.g., 4 seconds, 4.5 seconds, 5 seconds, 6 seconds, 7 seconds, etc.). The inhalation duration may be fixed or set (e.g., by a user, such as the physician, nurse, pharmacist, and/or the patient) or it may be variable. In some examples the inhalation duration may change to indicate that the minimum inhalation duration (e.g., of four seconds, 4.5 seconds, 5 seconds, etc.) has been reached, but that continuing inhalation is recommended. For example, the first indicator may be active for a minimum inhalation duration of 4 seconds using a first tone, color, etc., and may remain on for another 2-3 seconds but may change to a first optional/continuing indicator using a second tone, color, etc. For example, the nebulizer and/or dose guide apparatus may change from a red color to a yellow color or some other change to indicate inhalation may optionally continue.

The first indication (and/or the first optional/continuing indicator) may turn off automatically, e.g., after the patient has finished inhaling through the nebulizer and/or begun exhaling. The methods and apparatuses may include sensing inhalation and/or exhalation. For example the nebulizer and/or dose guide apparatus may include one or more sensors for detecting or deducing the start/stop of inhalation and/or exhalation. For example, a nebulizer may include one or more sensors for detecting flow or pressure at the mouthpiece. A flow sensor may be used to determine the start and/or stopping of inhalation through the mouthpiece. Any of these methods and apparatuses may include a controller (including one or more processors) that may perform these methods including triggering the first indicator, second indicator, etc.). The controller may analyze the sensor data to trigger the first and/or second indicators.

In general, the methods described herein may include instructing or guiding the patient to breathe in so that each breath is slow and long, breathing in until their lungs are as full as possible (e.g., breathe in as deeply as possible). Each inward breath in should last at least 4 seconds or longer as mentioned 109.

The second indicator, guiding the patient for the rapid (e.g., 3 seconds or less) exhalation may be triggered automatically as mentioned above (e.g., at the stop of inhalation) or based on a preset and/or settable timer. Generally, the method may include turning off the first indicator and/or activating the second indicator to guide exhalation 111. Because the exhalation phase is intended to be rapid and brief, the second indicator may include a “stop” indicator after the second (exhalation) duration of 3 seconds or less (e.g., 2 seconds), to alert the user to stop. For example, in some cases the second indicator may include a first phase from the start of exhalation to the end of the exhalation phase (2-3 seconds) 113 after which the second indicator may change to emphasize that the exhalation should be complete, for example by a change in the volume, tone, intensity, color, continuity (e.g., flashing) or the like. The second indicator may then turn off or otherwise stop 115.

Thus, during inhalation, the patient may be instructed and/or guided to breath out quickly through their nose, trying to finish breathing out within about 3 seconds (within about 2 seconds, within about 2-3 seconds, etc.). As discussed herein, this may direct the nebulized drug agent (e.g., mist) from the patient's lungs in the nose in the desired distribution, where it may be captured and give treatment to this area.

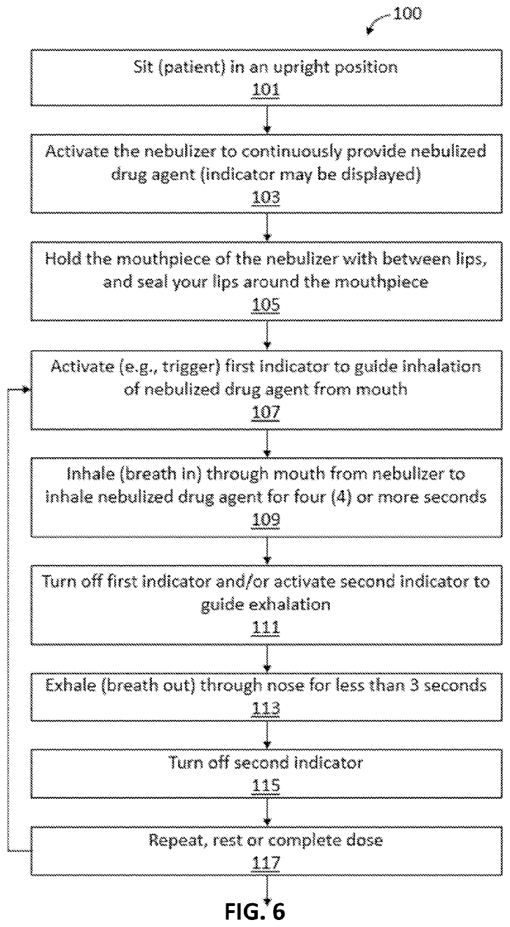

After the completion of the long inhalation/rapid exhalation, the patient may be instructed to either rest, e.g., breathe normally for one or more breaths, without the nebulizer, or to perform another cycle of long inhalation/rapid exhalation 117. For example, the patient may need to take a rest or if they have a cough or urge to cough. The patient may press the on/off button to stop the nebulizer. The treatment may be continued by once again pressing the on/off button on the nebulizer and/or dose guide apparatus to begin breathing in through the mouthpiece and out through the nose (repeating steps 107 to 117 in FIG. 6). The patient may take as many rests as needed.

Treatment may be continued until the desired (e.g., pre-set, user set, etc.) dose has been delivered. In some examples the treatment, including multiple cycles of long inhalation/rapid exhalation may be continued until the nebulizer and/or dose guide apparatus indicates the full treatment dose has been delivered. For example, the treatment may be continued until the nebulizer issues an alert (e.g., a beep and/or light flash), indicating that the treatment is complete. The device may turn off automatically. Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

As mentioned above, an apparatus may be configured to perform any of the methods described herein. For example, an apparatus may be configured as a nebulizer integrated with a (or forming the) dose guide apparatus. The nebulizer may be configured to emit the first indicator, such as a tone (e.g., beeping, etc.) or illuminating one or more LEDs (e.g., a countdown of LEDs), and the second indicator, such as a second tone or illuminating a different color or set of LEDs, etc. As mentioned above, the nebulizer may include one or more sensors for detecting and triggering the start of inhalation and/or exhalation to allow the device to count down and guide the user in inhaling and exhalating as described herein.

In some examples a separate dose guide apparatus may be used with a nebulizer. For example, the dose guide apparatus may be software. In some examples the software may be executed on a processor of a wearable or hand-held computing device, such as a smartphone.

These methods and apparatuses may be used with any type of nebulizer. For example, these apparatuses may be used with a jet nebulizer that uses a compressed gas to make an aerosol, an ultrasonic nebulizer, which forms the aerosol through high-frequency vibrations and/or a mesh nebulizer that passes liquid passes through a very fine mesh to form the aerosol. In particular, these methods may be used with continuous nebulizers that continuously form particles when on. Alternatively, these methods may be used with on-demand nebulizers.

In general, the methods and apparatuses described herein may apply to aerosol particles of a specific or predetermined size or size distribution. For example the particles of drug agent (MMAD) may be in the range from about 0.1 to about 200 microns (such as between about 1 to 10 microns, between about 2 to 7 microns, between about 2 to 20 microns, between about 10-40 microns, between about 20-60 microns, between about 30-70 microns, between about 40-80 microns, between about 50-90 microns, between about 60-100 microns, between about 70-110 microns, between about 80-120 microns, between about 90-130 microns, between about 100-150 microns, between about 125-200 microns, etc.). For example, particles containing the agent may have a particle or droplet size in the range from about 2 to 7 microns. In some examples the method described herein may be used with two distributions of particle sizes, including smaller and larger particle sizes.

Any appropriate drug agent may be used, including but not limited to drug agents that are mucosal trapping drug agents and/or immunotherapeutics. In general these drug agents may be drug agents for treating a respiratory disorder/disease, including disorders/diseases that are transmitted by respiration.

In particular, the drug agents described herein may include drug agents that are trapped within mucus, as described, e.g., in each of U.S. Pat. Nos. 10,829,543, 10,100,102, 10,793,623, U.S. patent application Ser. No. 16/982,682 (titled “COMPOSITIONS AND METHODS FOR INHIBITING PATHOGEN INFECTION” and filed Mar. 20, 2019), U.S. patent application Ser. No. 17/063,122 (titled “OPTIMIZED CROSSLINKERS FOR TRAPPING A TARGET ON A SUBSTRATE” and filed Oct. 5, 2020), and U.S. patent application Ser. No. 17/278,217 (titled “SYNTHETIC BINDING AGENTS FOR LIMITING PERMEATION THROUGH MUCUS” and filed Sep. 23, 2019), each of which is herein incorporated by reference in its entirety.

For example, the methods described herein may be particularly useful for delivering a dose of a drug agent that is configured to have an enhanced trapping potency in mucus, including but not limited to proteins (e.g., antibodies) that include one or more glycosylation patterns that enhance trapping in mucus. In some examples the drug agent may be a recombinant antibody comprising an oligosaccharide having a G0 glycosylation pattern comprising a biantennary core glycan structure of Manα1-6(Manα1-3)Manβ1-4GlcNAcβ1-4GlcNAcβ1 with terminal N-acetylglucosamine on each branch that enhances the trapping potency of the recombinant antibody in mucus. For example, the drug agent may be a recombinant antibody comprising a human or humanized Fc region, wherein the recombinant antibody comprises a population of antibodies in which at least 40% comprise an oligosaccharide having a G0 glycosylation pattern comprising a biantennary core glycan structure of Manα1-6(Manα1-3)Manβ1-4GlcNAcβ1-4GlcNAcβ1 with terminal N-acetylglucosamine on each branch that enhances the trapping potency of the recombinant antibody in mucus.

The techniques and procedures described or referenced herein can be employed using methodologies described in, for example, Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995). Methods and techniques for identifying amino acid sequences in constant and variable regions are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody. Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne et al. (2000) Bioinformatics 16: 741-742; Menne et al. (2000) Bioinformatics Applications Note 16:741-742; Wren et al. (2002) Comput. Methods Programs Biomed. 68:177-181; von Heijne (1983) Eur. J. Biochem. 133:17-21; von Heijne (1986) Nucleic Acids Res. 14:4683-4690).

### Examples

Various mAbs having different variable regions were used with the same general Fc region (e.g., SEQ ID No. 1), surprisingly showing that, despite the widely held belief that such proteins would be rapidly cleared within the lungs (within hours, if not minutes), instead, such mAb proteins, and in particular, those that were glycosylated to increase muco-trapping, persisted for days at therapeutically appreciable levels, when inhaled at safe and reasonable amounts during a single therapy, as described herein.

The results described herein are not limited to mAbs directed to pathogens. For instance, as the same Fc regions may be used with mAbs that are anti-inflammatory, and typically show the same clearance profiles as described herein. Anti-pathogen and anti-inflammation mAbs may be administered together for treating hospitalized patients, the methods and compositions described herein may be used for indications in which the anti-inflammatory mAbs are used alone.

The results described herein, as shown in the examples blow, are particularly surprising, given that increased muco-adhesion from having mAbs with a ‘muco-trapping’ Fc domain, e.g., by enriching for G0 glycosylation, has long been believed to lead to even faster clearance by mucosa, but surprisingly, the methods and compositions described herein may stay long enough in both the URT and LRT to sustain meaningful concentrations after 24 hrs.

### Example 1: Human IgG G1 Fc Region

A human IgG G1 Fc region (IN-006, a reformulation of regdanvimab), having an Fc region that is homologous with SEQ ID No. 1, was examined as part of a study was conducted in Australia. Study staff and participants were masked to treatment assignment, except for pharmacy staff preparing the study drug. The primary outcome was safety and tolerability. Exploratory outcomes were pharmacokinetic assessments of IN-006 in nasal fluid and serum.

Twenty-three participants were enrolled and randomized across two single dose and one multiple dose cohorts. There were no serious adverse events (SAEs). All enrolled participants completed the study without treatment interruption or discontinuation. All treatment-emergent adverse events were transient and graded mild to moderate in severity, without dose dependency. Nebulization was well tolerated and completed in a mean of 6 minutes for the high dose group. Mean nasal fluid concentrations of IN-006 in the multiple dose cohort were 921 μg/mL at 30 minutes after dosing, and 5.4 μg/mL at 22 hours. Mean serum levels in the multiple dose cohort peaked at 0.55 μg/mL at 3 days after the final dose.

IN-006 was well-tolerated and achieved concentrations in the respiratory tract orders of magnitude above its inhibitory concentration. These data support further clinical development of IN-006.

SARS-CoV-2, like many viruses that cause acute respiratory infections (ARIs), infects cells almost exclusively via the apical (luminal) side of the airway epithelium and also buds from infected cells primarily via the apical surface. Progeny virus must then travel through airway mucus to reach uninfected epithelial cells as the infection spreads from the upper respiratory tract (URT) to the lower respiratory tract (LRT) and the deep lung. Neutralizing monoclonal antibodies (mAbs) must therefore reach the airway lumen in sufficient quantities to effectively neutralize the virus and halt the infection.

mAbs distribute very poorly and slowly from the blood into the respiratory tract, with concentrations in the airways that are orders of magnitude lower than those in the serum following intravenous (IV) or intramuscular (IM) administration. Despite these limitations, the clinical experience to date has shown that mAbs that neutralize SARS-CoV-2 can be effective in treating infected individuals at high risk of severe COVID when administered by IV early in the course of the infection. Nevertheless, high doses of mAb are generally required to do so, reducing the drug supply available. Delayed distribution into the lung also limits the treatment window for preventing severe COVID.

Nebulization has been used to deliver protein therapeutics (e.g., Pulmozyme) directly to the lungs, enabling dosing within minutes. Importantly, direct inhaled delivery can achieve far higher concentrations of drug in the lungs than can be achieved by IV or IM administration and does so within minutes. Since the pattern of deposition along the respiratory tract is largely determined by the aerosol droplet size, it is possible to use a nebulizer that generates a broad aerosol size distribution to deliver drug throughout the entire respiratory tract, from the nasal turbinates in the URT, through conducting airways in the LRT, to the deep lung. Thus, nebulized delivery is likely the fastest method to achieve high inhibitory concentrations of mAb in the airway fluids. Nebulization also enables convenient self-dosing at home, reducing the burden on patients and on the healthcare infrastructure associated with systemic delivery.

IN-006 is reformulation of regdanvimab configured as described herein specifically for nebulized delivery, as an inhaled treatment for COVID-19. Regdanvimab, an IV dosed human IgG1 mAb directed against the SARS-COV-2 spike protein receptor binding domain (RBD), is approved in the European Union for adults with COVID-19 who do not require supplemental oxygen and who are at increased risk of progressing to severe COVID-19.

A double-blind, placebo-controlled, first-in-human, ascending-dose pharmacokinetic and safety study was conducted in a Phase 1 unit in Melbourne Australia. The study was carried out according to the International Council for Harmonisation Good Clinical Practice guidelines and in compliance with local regulatory requirements and was approved by The Alfred Hospital Office of Ethics and Research Governance, Melbourne, VIC, Australia. Informed consent was obtained in advance of all study-related procedures. Eligible participants were enrolled sequentially into three cohorts: a single low dose cohort (30 mg), a single high-dose cohort (90 mg), and a multiple high-dose cohort (seven daily 90 mg doses). For each single dose cohort, a sentinel pair (with one active and one placebo recipient) was initially dosed, followed by a two-day safety monitoring period prior to the dosing of the remainder of the cohort. Advancing to subsequent cohorts was done after review of safety parameters seven days after dosing of the preceding cohort. FIG. 3 shows a diagram of the study structure and times of pharmacokinetic evaluations.

Eligibility criteria required that participants be adults 18-55 years of age with a body-mass index of 18-32 kg/m² who were in good health as judged by medical history, physical exam, clinical chemistry and hematology assessments, electrocardiogram, forced expiratory volume in one second (FEV₁)≥90% predicted, and negative serology for HBsAg, HCV and HIV antibodies. Participants were required to be non- or light smokers. The FEV₁ threshold was changed to 80% predicted after enrolling the first 7 participants. Participants were excluded for known or suspected symptomatic viral infection or signs of active pulmonary infection or pulmonary inflammatory conditions within 14 days of dosing initiation, a history of airway hyperresponsiveness, angioedema, anaphylaxis, or a positive alcohol breathalyzer test and/or urine drug screen for substances of abuse. During recruitment of the 7 participants comprising the first single dose cohort, participants who had received a COVID-19 vaccine were excluded. However, due to rapidly increasing local vaccine availability and uptake, this criterion was modified to exclude only those vaccinated within two weeks of initial dosing, or those with plans to be vaccinated within two weeks after completion of dosing.

The primary endpoint for the trial was the safety and tolerability of IN-006. This was assessed by monitoring treatment-emergent adverse events, pre- and post-dose vital signs, ECG, FEV₁, SpO₂, hematology and chemistry safety blood tests, and physical examinations. Follow-up continued for 28 days, with assessments on the days indicated in FIG. 3. Exploratory outcomes were drug levels in nasal fluid and serum pre dose and at intervals post dose. A randomization schedule was prepared using validated software (SAS) by statistical team members who had no responsibility for monitoring and data management of this study, with provisions for each sentinel pair to include one active and one saline placebo assignment, and for the overall ratio of active to placebo assignment of each cohort to be 3:1. The randomization code was held by unblinded pharmacy staff who prepared the doses in matching syringes with identical appearances for loading into the nebulizer by clinical staff.

IN-006 was produced under Good Manufacturing Practices (GMP) and supplied as a liquid formulation in glass vials from the manufacturer. IN-006 was provided in a syringe to be loaded into the InnoSpire Go vibrating mesh nebulizer (Koninklijke Philips N.V.). Placebo participants received an identical syringe containing saline instead of IN-006. Participants were instructed to breathe in slowly through the nebulizer mouthpiece and to breathe out through their nose. Nasal fluid was obtained by rotating a flocked swab (Copans Cat. #56380CS01) for 10-15 seconds at mid-turbinate depth (4-5 cm). Sampling alternated between right and left nostrils during sequential sample collection timepoints. The amount of nasal fluid sample collected by each individual swab was determined by comparing pre- and post-weights. This was achieved by weighing the sample-containing swab and sample tube before and after it was incubated in buffer for extraction, rinsed, and oven dried. Sampling times for nasal fluid and serum are shown in FIG. 3. Vital signs and FEV₁ were measured before nebulization and 15 and 30 minutes after completion. IN-006 concentration was measured in the human serum and nasal fluid. Sample size was chosen according to conventions for Phase 1, first in human studies. Formal sample size and power calculations were not performed. Continuous variables were summarized using descriptive statistics including number of non-missing observations, mean, SD, median, minimum, and maximum values. Categorical variables were summarized with frequency counts and percentages. Placebo recipients in different cohorts were pooled. The safety analysis included all randomized participants who received any dose of study drug. The pharmacokinetic population included all participants who received any dose of IN-006. No inferential statistical tests were conducted. Serum PK parameters of IN-006 were determined using Phoenix WinNonlin version 8.3.

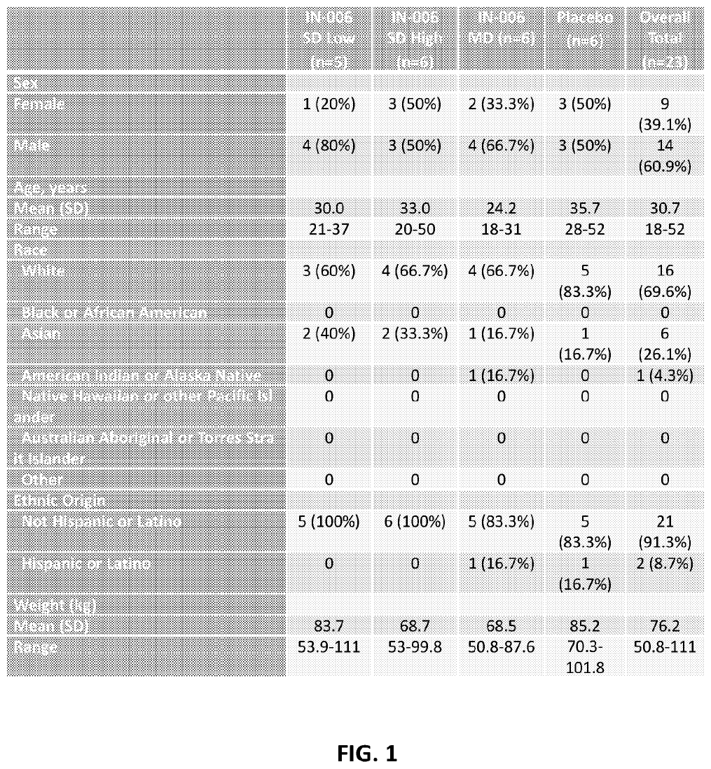

From among 102 adults who were screened, 23 participants were sequentially assigned to one of three cohorts. The first participant was randomized on Sep. 22, 2021, and the last participant visit was on Dec. 29, 2021. Of these participants, 17 were randomly assigned to receive IN-006, and 6 were randomly assigned to receive placebo. All 23 participants received their assigned treatment as intended and completed the final study visit on Study Day 29. The study was completed on Dec. 29, 2021. Participant flow is diagrammed in FIG. 3, and participant demographics are listed in Table 1 (FIG. 1).

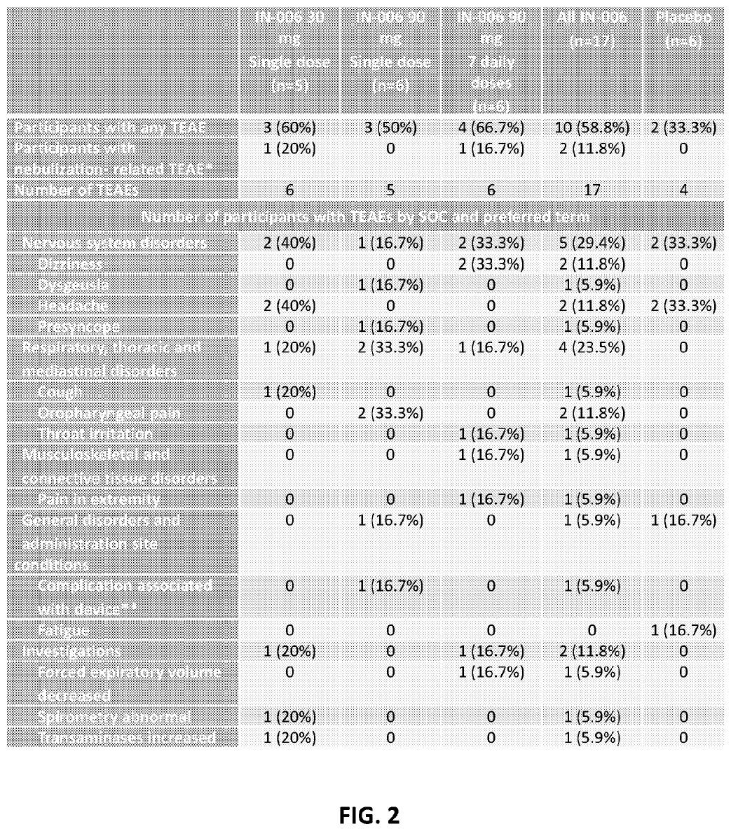

Treatment emergent adverse events (TEAEs) are listed in Table 2 (FIG. 2). Nebulization of IN-006 was well-tolerated and completed in an average of 6 minutes for the 90 mg dose (range 4-9 minutes). Eight (53.3%) of the 15 participants included in the single ascending dose cohorts experienced at least 1 TEAE (6 receiving IN-006, 2 receiving placebo). The most frequently reported TEAEs were headache (4/15; 26.7%) and oropharyngeal pain (2/15; 13.3%). All but 1 TEAE were mild. One participant receiving IN-006 low dose (30 mg) experienced a moderate event (increased transaminases on Day 29), which was not considered to be related to study drug by the investigator. Three (3/15; 20.0%) participants experienced at least 1 TEAE considered related to study drug by the investigator. These events included headache, cough, and oropharyngeal pain. All 3 related TEAEs were mild and resolved. There was no evidence of a dose-related effect.

In the multiple dose cohort, no TEAEs were reported in participants receiving placebo. Among the six participants receiving IN-006, four (66.7%) participants experienced at least one TEAE. The most frequently reported TEAE was dizziness (2/6; 33.3%). All but one TEAE were mild. One single participant receiving IN-006 experienced a moderate event (pain in extremity). The event was considered unlikely to be related to study drug by the investigator. Two (33.3%) participants experienced at least one TEAE considered related to study drug by the investigator. These drug-related TEAEs were dizziness and decrease in FEV₁: the latter was noted 15 minutes after nebulization, was not associated with symptoms or abnormal vital signs, resolved within 15 minutes, and did not recur with subsequent doses. Both events were mild.

No severe TEAEs, SAEs, or TEAEs leading to discontinuations were reported in either the single dose or multiple dose cohorts. There were no unexpected safety signals.

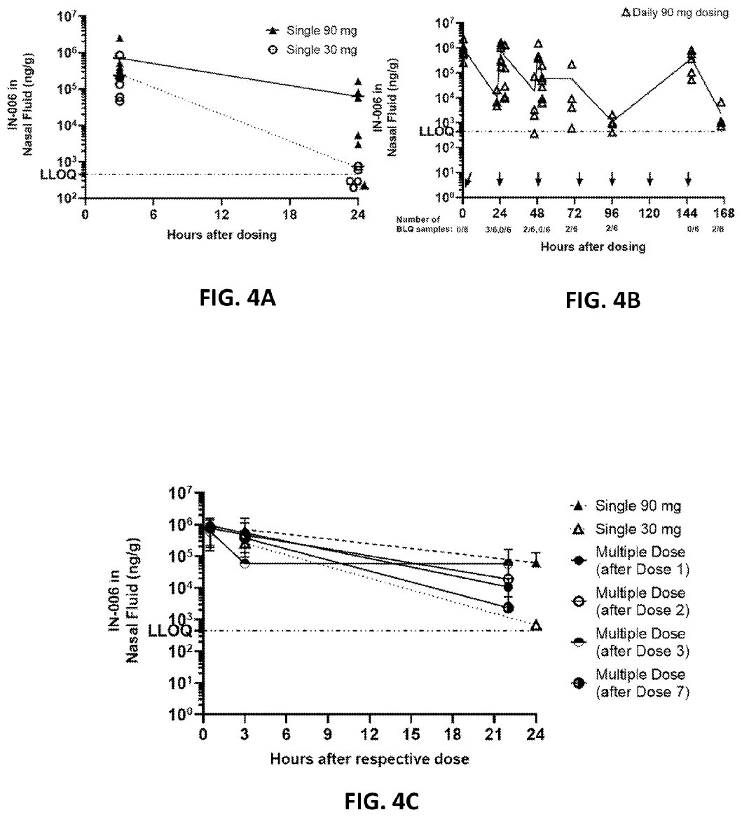

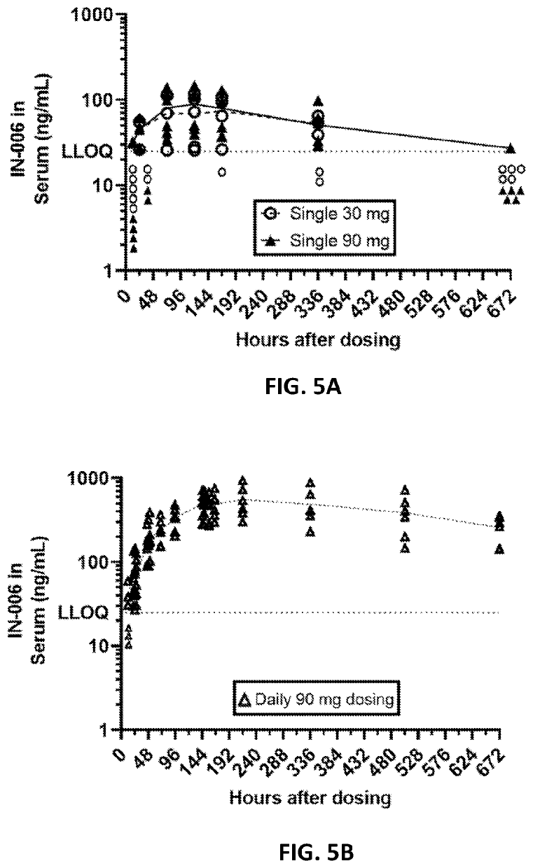

For single dose cohorts, the mean nasal concentrations were 261 μg/g and 710 μg/g for the 30 mg and 90 mg dose, respectively, measured 3 hrs after dosing; these values are consistent with a 3-fold increase in the dose administered. In the multiple dose cohort, the repeated dosing provided additional opportunities for more nasal concentration measurements across more time points. The nasal concentrations measured 30 mins after dosing on Days 1, 2 and 3 averaged 773 μg/g, which is higher than the concentrations measured 3 hrs after dosing (405 μg/g). This indicates that peak exposure occurred immediately after dosing, and the nasal concentrations had appreciably reduced by 3 hours post-dose. There was minimal intranasal accumulation, as the concentrations of IN-006 measured 22 hrs after dosing were <2% of the concentrations immediately following dosing (FIGS. 4A-4C). The difference in nasal concentrations between 30 mins and 22 hrs post-dose suggest the interval spanned ˜6-7 half lives, and the difference between 30 mins and 3 hrs post-dose was roughly half. Both are consistent with an intranasal half-life of roughly 3-4 hours, markedly longer than the timescale of mucociliary clearance transit time estimates of ˜5-15 minutes from saccharin transit time tests.

As shown in FIGS., 5A-5B, serum concentrations of IN-006 were detectable by 12 hrs following nebulization at the 90 mg dose and continued to rise through 120 hrs after a single dose (cohorts 1 and 2), or through 216 hrs following the first dose in those who received multiple doses (cohort 3). The elimination half-life of IN-006 in the serum was estimated to be ˜253, 292, and 402 h in the 30 mg single dose, 90 mg single dose, and the 7 daily 90 mg dose cohorts, compatible with the previously estimated elimination half-life of regdanvimab from the serum following intravenous administration (288 h). Although the serum concentrations of IN-006 were markedly lower than those in the nasal fluid (Serum Cₘₐₓ of 0.52 μg/mL, compared to 990 μg/mL in nasal fluid), they were still significantly greater than the IC₅₀ of IN-006 (˜0.01 μg/mL).

A longstanding dogma has been that it is highly challenging to stably nebulize mAbs (see, e.g. Respaud, R., et al., Nebulization as a delivery method for mAbs in respiratory diseases. Expert Opin Drug Deliv, 2015. 12(6): p. 1027-39; Mayor, A., et al., Inhaled antibodies: formulations require specific development to overcome instability due to nebulization. Drug Deliv Transl Res, 2021. 11(4): p. 1625-1633; Bodier-Montagutelli, E., et al., Protein stability during nebulization: Mind the collection step! Eur J Pharm Biopharm, 2020. 152: p. 23-34; and Bodier-Montagutelli, E., et al., Designing inhaled protein therapeutics for topical lung delivery: what are the next steps? Expert Opin Drug Deliv, 2018. 15(8): p. 729-736.), and that biologic drugs would be quickly eliminated from the respiratory tract either by systemic absorption, physical mucociliary clearance, or degradation by alveolar macrophages, making it difficult to sustain therapeutic concentrations. See, e.g., Loira-Pastoriza, C., J. Todoroff, and R. Vanbever, Delivery strategies for sustained drug release in the lungs. Adv Drug Deliv Rev, 2014. 75: p. 81-91, and Suri, R., The use of human deoxyribonuclease (rhDNase) in the management of cystic fibrosis. BioDrugs, 2005. 19(3): p. 135-44.

Surprisingly, as described herein, IN-006, a reformulation of regdanvimab for nebulized delivery, was safe and well tolerated in healthy adults, with minimal side effects and high concentrations of drug recovered from nasal and serum samples, and was able to sustain therapeutic concentrations. This may be due in part to the glycosylation of the therapeutic antibody, as described herein in combination with the administration technique. The treatment was easily self-administered by all participants and was completed within minutes. Encouragingly, the concentrations of IN-006 measured in nasal secretions were well above its IC₅₀, even 22-24 hrs after dosing. In the multiple dose cohort that received seven daily 90 mg doses, a mean nasal fluid IN-006 concentration of 920 μg/mL 30 minutes was observed after the initial dose and retained a mean concentration of 5.4 μg/mL 22 hours later, prior to receiving a second dose. The ability to maintain mAb concentrations that ranged from 3-7 orders of magnitude higher than the IC₅₀ for regdanvimab and other COVID mAbs against susceptible variants (˜4-20 ng/mL) strongly support our proposed once-daily dosing regimen. Since SARS-CoV-2 infection and replication initiates in the upper respiratory tract, our efficient delivery of IN-006 to the nasal passages suggests it may provide a highly effective treatment for early COVID-19 that allows earlier resolution of the infection and reduced risk of progression to severe COVID.

Although mAbs have proven to be effective therapeutics for COVID-19, the necessity for administration by IV, IM, or SC routes has limited the scope of their use in clinical practice. The requirement for infusion centers and post-dosing observation for intravenous administration have severely limited the number of patients that have received treatment, and greatly increased costs. IM injections, although shortening administration time, are limited by the volume that can be administered per injection (˜5 mL), which in turns limit the dose of mAb that can be dosed per injection, and can be painful when maximum injection volumes are used. In contrast, nebulized delivery using a handheld nebulizer enables the convenience of at-home dosing, and only takes minutes to complete. Furthermore, IV, IM, and SC routes provide mAb to the airway lining fluid only after a delay of one or more days, and even then only achieve airway concentrations that are a fraction of the concentrations in plasma. For instance, in a recent clinical trial of the anti-influenza mAb CR6261 given as a single 50 mg/kg dose IV, the peak nasal concentration was not achieved until 2 days after infusion, and the peak nasal concentration of 0.597 μg/mL was ˜10-fold lower than the concentrations observed for IN-006 at the trough of our daily dosing (˜5.4 μg/mL), despite the much lower total dose of IN-006 compared to CR6261 (90 mg IN-006 vs. ˜2,000-4,000 mg CR6261). The increased convenience, more efficient pulmonary delivery, and superior pharmacokinetics may make inhalation the preferred route of mAb delivery for treating acute respiratory infections.

COVID-19 is predominantly a respiratory tract infection, however currently available treatments are administered by systemic dosing. Described herein are methods and compositions of mAbs constructed using a human IgG that binds to the spike protein of SARS-CoV-2. The methods and compositions described herein may provide inhaled delivery of a muco-trapping monoclonal antibody (including a Fc region with G0 glycosylation) that may provide a more convenient and effective treatment for COVID-19. The results describe in Example 1 show the safety, tolerability, and pharmacokinetics of one example of a Human IgG G1 Fc region (IN-006, a reformulation of regdanvimab, an approved intravenous treatment for COVID-19) that may be used for nebulized delivery by a handheld nebulizer.

Serious disease due to SARS-Cov-2 is accompanied by the spread of the virus from the site of the initial upper respiratory tract infection to the deep lung. Unfortunately, the exact timing of such spread is likely to be highly variable between individuals. Indeed, there is evidence suggesting viruses can already reach the LRT even during early stages of disease, around when symptoms emerge. Similarly rapid spread of the infection to the LRT is likely also frequent for other viruses such as influenza. Thus, dosing to both the URT and LRT, rather than focusing exclusively on the URT (e.g., via nasal sprays), may be important to broaden the treatment window and reduce the risk of COVID-induced pneumonia and hospitalization. While IN-006 levels in the LRT were not directly measured in this study, the appreciable serum concentrations, and the delayed serum Tmax, both strongly suggest IN-006 is efficiently delivered into the LRT and the deep lung. Indeed, in a toxicokinetic multiple dose nebulization study of IN-006 in rats, IN-006 concentrations in airway fluid exceeded the serum concentrations by ˜100-fold. Efficient delivery into the LRT is a direct consequence of our design requirement for the vibrating mesh nebulizer. The droplet sizes generated by the nebulizer (the fine particle fraction, i.e. droplets <5 μm, and particularly those <2.5 um) were intentionally selected to deliver mAbs throughout the LRT and deep lung. Furthermore, the fact that a slow steady rise of serum concentrations was observed in single dose cohorts over ˜4 days, and peak serum concentrations in the multiple dose cohort of ˜9 days (or 2 days after last dose), implies that high levels of IN-006 are sustained in the deep lung for at least that duration, i.e. over many days. Assuming a similar ratio of 100:1 airway fluid-to-serum concentrations in humans as observed in rats, the mean human serum concentrations of 200 ng/mL at 2 days after first dose and 550 ng/mL at Day 9 should translate to pulmonary concentrations on the order of 50 μg/mL, which is >3 orders of magnitude above the IC₅₀, and comparable to the serum concentrations achieved with some IV/IM-dosed mAbs. The very high mAb levels sustained relative to the intrinsic activity of the mAb (IC₅₀) may continue to provide effective treatment against variants, even in the presence of appreciable genetic drift, and may reduce the risk of inducing viral escape. It also suggests that shorter duration therapy, perhaps as short as a one-time dosing, could afford appreciable protection against hospitalization.

Despite significant extrapulmonary manifestations of severe COVID-19, and despite frequent detection of SARS-CoV-2 RNA in blood, infectious SARS-CoV-2 is rarely detected in the blood of infected patients, suggesting that extrapulmonary disorders can be caused by indirect factors such as the inflammatory response rather than extrapulmonary viral infection. Nonetheless, it is reassuring to observe that serum levels of IN-006 achieved after nebulized delivery were in excess of IC₅₀ by at least one order of magnitude.

Regdanvimab (administered IV) was shown to be highly efficacious for preventing severe COVID-19 in a global Phase 3 study, leading to its formal approval in Republic of Korea and European Union (EMEA/H/C/005854) for preventing severe disease in patients presenting with mild to moderate COVID-19, and emergency use authorization (EUA) or conditional marketing authorization in several additional countries worldwide. IN-006 may be combined with a second potent neutralizing mAb to create a mAb cocktail that possesses potent binding activity against every variant tested to date. The surprisingly long airway retention of IN-006 observed here may be used for virtually and mAb including the Fc region (e.g., SEQ. ID NO. 1, SEQ. ID NO. 2, SEQ. ID NO. 3, SEQ. ID NO. 4). IN-006, a reformulation of regdanvimab for inhaled delivery, was found to be safe and well tolerated in healthy participants at single doses of 30 mg and 90 mg, as well as seven consecutive daily doses of 90 mg. Nebulization resulted in IN-006 levels in nasal fluids, and likely the lungs, that are orders of magnitude above the inhibitory concentrations of sensitive SARS-CoV-2 variants within 30 minutes, and the continued rise of serum concentration for days after dosing implied substantial lasting IN-006 levels in the lungs.

### Example 2

The methods described herein can be used to provide sufficient levels of antibody in both the upper respiratory pathway and the lower respiratory pathway.

Subjects will receive a first dose of IN-006 or placebo via a nebulizer on Dosing Day 1. Placebo will be 0.9% normal saline and will be administered via a nebulizer in an identical manner as IN-006. Subjects will receive a second dose of IN-006 or placebo via a nebulizer on at least one of Dosing Day 3-Dosing Day 8. Placebo will be 0.9% normal saline and will be administered via a nebulizer in an identical manner as IN-006. Measurement of antibody will be performed using bronchoscopy with bronchoalveolar lavage (BAL) before and 2 weeks after treatment. Bronchoalveolar lavage will be performed infusions of warmed sterile PBS into a segmental middle-lobe bronchus with the bronchoscope. The fluid will be recovered by gentle suction and collected in a sterile container. It will be filtered through a sterile 100-μm mesh to remove mucus and cell debris and analyzed using the methods described herein.

### Example 3

The methods described herein can be used to provide sufficient levels of at least two therapeutic agents in both the upper respiratory pathway and the lower respiratory pathway. Subjects may receive a first dose of IN-006 and a second therapeutic agent (e.g., non-mAb) or placebo via a nebulizer on Dosing Day 1. Placebo will be 0.9% normal saline and will be administered via a nebulizer in an identical manner as IN-006. Nasal swabs will be taken on at least one Dosing Day 3-Dosing Day 8 and measure for levels of antibody. Subjects will receive a second dose of IN-006 or placebo via a nasal sprayer on at least one of Dosing Day 3-Dosing Day 8. Placebo will be 0.9% normal saline and will be administered via a nebulizer in an identical manner as IN-006.

### Example 4

The methods described herein can be used to provide sufficient levels of antibody in at least one or both of the upper and lower respiratory tracts with 1× (once) or 2× (twice) per day dosing.

Subjects will receive a first dose of antibody (e.g., IN-006 or other) or placebo via a nebulizer at time 0 on Dosing Day 1. The antibody may be an antibody glycosylated with the G0 glycosylation pattern. Placebo will be 0.9% normal saline and will be administered via a nebulizer in an identical manner as antibody (e.g., IN-006 or other). A first cohort of Subjects will receive a second dose of antibody (e.g., IN-006 or other) or placebo via a nebulizer at 12 hours after the first dose. Placebo will be 0.9% normal saline and will be administered via a nebulizer in an identical manner as antibody (e.g., IN-006 or other). Measurement of antibody will be performed using bronchoscopy with bronchoalveolar lavage (BAL) before, immediately after dosing, at 12 hours (prior to the second dose), and at 24 hours. Bronchoalveolar lavage will be performed infusions of warmed sterile PBS into a segmental middle-lobe bronchus with the bronchoscope. The fluid will be recovered by gentle suction and collected in a sterile container. It will be filtered through a sterile 100-um mesh to remove mucus and cell debris and analyzed using the methods described herein.

TABLE 3

| Proposed Dosing | Predicted Peak | Predicted Trough |
|---|---|---|
| Regimen | ug/mL) | (ug/mL) |
| 90 mg dose, 1x/day: | 900 | 7.8 |
| 30 mg dose, 2x/day: | 300 | 27.9 |
| 15 mg dose, 2x/day: | 150 | 13.9 |

The methods and compositions described herein may allow delivery of the inhaled therapeutic mAb, including those having the G0 glycosylation pattern, which are otherwise expected to be cleared within minutes based on published work, to achieve sustained high concentrations for 24 hours or more, allowing once daily or twice daily dosing with relatively low (and therefore affordable) concentrations. Thus, the dose may deliver the therapeutic mAb so that sufficient levels are maintained until the next dosing (i.e. trough concentration).

As described herein, the peak concentration achieved in the upper respiratory tract scales with the amount of mAb-inhaled (e.g., going from 30 mg to 90 mg inhaled resulted in ˜3× increase). Next, the rate of clearance was surprisingly dose independent, with comparable clearance rates of the 30 mg and 90 mg once dose, as well as comparable clearance between the 90 mg once vs. different days of the repeated 90 mg dose. The ‘muco-trapping’ mAbs (those having the G0 glycosylation pattern) are not cleared within minutes as previously suggested (e.g., faster than 30 minutes) for the turnover rate of the nasal secretions in the nasal turbinate, but rather, have a half-life in the range of 3.5-4 hrs.

This allows for a dosing regimen to not only achieve sufficient excess mAb dose above IC50 immediately following dosing, but also allows sufficient mAb to be retained at trough immediately before the next dosing. Thus, the dose for different mAbs may be selected, depending on their potencies, to achieving sufficient excess of the concentrations relative to their inherent potencies (for example, maintain 10-100× above IC50 of a mAb where the IC50 is ˜100 ng/mL). For example, the methods and compositions described herein indicate that twice per day, 15 mg dose each time provide a significant dose.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims. The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1.-34. (canceled)

35. A method of treating a subject having, or at risk of having, a respiratory disorder, the method comprising administering, by inhalation, to the subject a formulation comprising a therapeutic human IgG monoclonal antibody (mAb) comprising a population of antibodies in which at least 40% are glycosylated with a G0 glycosylation pattern comprising a biantennary core glycan structure of Manα1-6(Manα1-3)Manβ1-4GlcNAcβ1-4GlcNAcβ1, wherein administering comprises administering a dose of 0.02 μmol or more of the therapeutic human mAb no more than twice per day to achieve a concentration of greater than 20 ng/mL for the therapeutic human mAb in an upper respiratory tract (URT) and a concentration of greater than 100 ng/mL in a lower respiratory tract (LRT) for 12 hours or more after the dose administration.

36. The method of claim 35, wherein administering comprises administering the dose no more than once per day.

37. The method of claim 35, wherein the therapeutic antibody comprises at least 50% of the G0 glycosylation pattern.

38. The method of claim 35, wherein the therapeutic antibody comprises an Fc sequence that is at least 80% homologous to the sequence of SEQ ID NO. 1.

39. The method of claim 35, wherein the subject is an adult.

40. The method of claim 35, wherein the therapeutic antibody comprises regdanvimab.

41. The method of claim 35, wherein the dosing administration comprises a dosing cycle of twice per day over a period of two days to seven days.

42. The method of claim 35, wherein the dosing administration comprises a dosing cycle of every second day, every third day, or every fourth day.

43. The method of claim 35, wherein administering the dose comprises administering at least 10 mg of the therapeutic human mAb.

44. The method of claim 35, wherein administering the dose comprises administering between about 10 mg and 100 mg of the therapeutic human mAb.

45. The method of claim 35, wherein administering comprises sustaining a release of the therapeutic human mAb into the subject's blood from the LRT over multiple days.

46. The method of claim 35, wherein administering comprises sustaining release of the therapeutic human mAb into the subject's lungs and blood over at least two days.

47. The method of claim 35, wherein the therapeutic antibody formulation further comprises a pharmaceutically acceptable diluent, excipient, and/or carrier.

48. The method of claim 35, wherein the therapeutic antibody formulation further comprises one or more of: citrate, arginine, mannitol, sorbitol, or trehalose.

49. The method of claim 35, wherein administering comprises administering the therapeutic antibody formulation to the subject via a nebulizer.

50. The method of claim 35, wherein administering comprises administering the therapeutic antibody formulation to the subject via a vibrating mesh nebulizer.

51. The method of claim 35, wherein administering comprises administering the therapeutic antibody formulation via inhalation or via direct instillation into an upper airway.

52. The method of claim 35, wherein administering comprises self-administration of the therapeutic antibody formulation by the subject.

53. The method of claim 35, wherein the respiratory disorder comprises a lower airway disorder.

54. The method of claim 35, wherein the respiratory disorder comprises an upper airway disorder.

55. The method of claim 35, wherein the respiratory disorder comprises an inflammatory disorder.

56. The method of claim 35, wherein the therapeutic antibody binds to a respiratory virus and the respiratory virus comprises a coronavirus.

57. The method of claim 35, wherein the therapeutic antibody binds to a respiratory virus and the respiratory virus comprises severe acute respiratory syndrome coronavirus 2 (SARS-CoV-2).

58. The method of claim 35, wherein the therapeutic antibody binds to a respiratory virus and the respiratory virus comprises respiratory syncytial virus (RSV).

59. The method of claim 35, wherein the therapeutic antibody binds to a respiratory virus and the respiratory virus comprises one or more of: influenza virus, metapneumovirus, parainfluenza virus, or coronavirus.

60. The method of claim 35, wherein the therapeutic antibody binds to a respiratory virus and the respiratory virus comprises a paramyxovirus.

61. The method of claim 35, wherein the formulation comprises a second therapeutic agent in addition to the therapeutic antibody.

62. The method of claim 35, wherein the formulation comprises the therapeutic human mAb and a second therapeutic antibody, and the therapeutic human mAb and the second therapeutic antibody bind to the same virus, but do not compete for binding to the virus.

63. The method of claim 35, wherein the formulation comprises a second therapeutic antibody in addition to the therapeutic human mAb, further wherein the therapeutic human mAb and the second therapeutic antibody bind to different viruses.

64. The method of claim 35, wherein the formulation comprises a biologic in addition to the therapeutic human mAb.

65. A method of treating a subject having, or at risk of having, a respiratory disorder, the method comprising maintaining a concentration of greater than 20 ng/ml of a therapeutic human IgG monoclonal antibody (mAb) that binds to a respiratory virus in an upper respiratory tract (URT) of the subject and a concentration of greater than 100 ng/ml in a lower respiratory tract (LRT) of the subject for more than 12 hours after a dose by administering, by inhalation, to the subject the dose of the therapeutic human IgG monoclonal antibody (mAb) comprising a population of antibodies in which at least 40% are glycosylated with a G0 glycosylation pattern comprising a biantennary core glycan structure of Manα1-6(Manα1-3)Manβ1-4GlcNAcβ1-4GlcNAcβ1, wherein administering the dose comprises administering 0.02 μmol or greater of the therapeutic human mAb no more than twice per day.

66. A method of treating a subject having, or at risk of having, a respiratory disorder, the method comprising maintaining a concentration of greater than 25 ng/ml of a therapeutic human IgG monoclonal antibody (mAb) that binds to a respiratory virus in an upper respiratory tract (URT) of the subject and a concentration of greater than 25 ng/ml in a lower respiratory tract (LRT) of the subject for more than 12 hours after the dose by administering, by inhalation, to the subject the dose of a formulation comprising the therapeutic human IgG monoclonal antibody (mAb) that binds to the respiratory virus, wherein administering comprises administering 0.02 μmol or greater of the therapeutic human mAb no more than twice per day.


SEQUENCE LISTING (4 sequences)

SEQ ID NO: 1 - protein, 220 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 216 aa, Homo sapiens
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 3 - protein, 216 aa, Homo sapiens
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 4 - protein, 217 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK